(19) United States
(12) Patent Application Publication
(10) Pub. No.: US 2025/0145928 A1
(43) Pub. Date: May 8, 2025

(54) BIOREACTOR

(71) Applicant: Ivy Farm Technologies Limited, Oxford (GB)

(72) Inventors: Akinlolu ODELEYE, Oxford (GB); Robin NYLAND, Oxford (GB)

(21) Appl. No.: 18/837,295

(22) PCT Filed: Feb. 8, 2023
(86) PCT No.: PCT/GB2023/050282

§ 371 Date: Aug. 9, 2024

(30) Foreign Application Priority Data
Feb. 11, 2022 (GB) 2201818.8

(51) Int. Cl. C12M 1/00; C12M 1/12; C12N 5/077

(52) U.S. Cl. CPC C12M 29/00; C12M 25/18; C12N 5/0658

(57) ABSTRACT

There is provided a method of culturing cells comprising: providing a bioreactor, wherein the bioreactor contains a particulate substrate arranged as a fixed bed; seeding the particulate substrate with myoblasts, myocytes, and/or myotubes; culturing the cells by flowing cell culture medium through the fixed bed such that the fixed bed conditions are maintained. Also provided is a flow distributor for a bioreactor comprising: a first surface and a second surface, wherein the first surface is on the opposite side of the flow distributor to the second surface, and wherein the flow distributor has a central axis extending through the flow distributor; and a plurality of channels within the flow distributor, wherein the plurality of channels each have an inlet and an outlet.

### FIELD

The present invention relates to a method of culturing cells. The present invention also relates to a flow distributor and a bioreactor system that can incorporate the flow distributor.

### BACKGROUND OF THE INVENTION

There are concerns about the sustainability of traditional farming practices relating to the rearing of livestock. These traditional farming practices are energy intensive, land intensive and utilise a large amount of antibiotics. A possible approach for addressing these concerns is culturing of meat in a bioreactor. This requires far less energy and land and is a desirable approach for feeding the world's growing population.

However, the success of meat culture depends on the commercial viability of the culture process. The present invention provides means for increasing meat culture viability and efficiency.

### SUMMARY OF THE INVENTION

The present invention provides a method of culturing cells comprising: providing a bioreactor, wherein the bioreactor contains a particulate substrate arranged as a fixed bed; seeding the particulate substrate with myoblasts, myocytes, and/or myotubes; culturing the cells by flowing cell culture medium through the fixed bed such that the fixed bed conditions are maintained.

This approach ensures the careful culturing of shear sensitive adherent cells, while still allowing for the flow of cell culture medium to promote cell growth. Thus, the present invention is particularly advantageous for use with cell types that are sensitive to the forces experienced by cells in a bioreactor, such as shear stress.

### DETAILED DESCRIPTION OF THE INVENTION

The inventors found that myoblasts, myocytes, and myotubes are highly susceptible to damage and detachment from substrates in bioreactors. Therefore, in the present invention, it is preferred that the cells seeded onto the particulate substrate comprise myoblasts, myocytes, myotubes, or a combination thereof. Preferably, the cells seeded onto the particulate substrate comprise myoblasts. During cell culture, the cells or a portion of the cells may differentiate. Thus, the cultured cells may comprise myoblasts, myocytes, myotubes, or a combination thereof. Preferably, the cultured cells comprise myocytes and/or myotubes.

The cells may be primary cells or cell lines. Exemplary cell lines include C2C12 cells (a murine myoblast cell line) and L6 cells (a rat myoblast cell line).

Although the aforementioned cell types are preferred for use in the present invention, any adherent cell type (i.e. any cell type that grows on a substrate, also known as anchorage-dependent cells) may be used. For example, embryonic stem cells (ESCs), induced pluripotent stem cells (iPSCs), mesenchymal stem cells (MSCs), muscle stem cells (muscle satellite cells), or combinations thereof may be used to seed the bioreactor.

Cells for use in the invention may be of any animal origin. However, typically, the cells are not human cells. Preferably, the cells are not produced through the destruction of human embryos. The cells are typically cells of non-human animals, such as non-human mammals, birds, fish, crustaceans, molluscs, reptiles, amphibians, or insects.

Exemplary non-human mammals include those in the genera Bovinae, Camelidae, Canidae, Caprae, Cervidae, Felidae, Equidae, Lagomorphs, Macropodidae, Oves, Rodents, or Suidae. The cells may be cells of livestock or poultry. The cells may be porcine, bovine, ovine, caprine, avine, or piscine.

In the present invention, the cells are preferably seeded when the particulate substrate is in the bioreactor. Alternatively, the cells may be seeded before the particulate substrate is introduced into the bioreactor.

The bioreactor that can be used with the present invention is any suitable bioreactor for culturing adherent cells. In general terms, a bioreactor is a vessel that can support the maintenance and growth of cells. This is also referred to as the culture of cells.

The bioreactor houses the substrate along with cell culture medium, and optionally cells. The cell culture medium may contain nutrients and growth factors within a fluid that supports the maintenance, proliferation, and growth of the cells. An exemplary culture medium is Dulbecco's Modified Eagle Medium (DMEM). Cell culture medium for use in the invention may comprise serum or may not comprise serum. The cell culture medium may contain factors promoting cell differentiation. The cells that are adhered on the substrate are submerged in the cell culture medium. The cell culture medium may be moved around the bioreactor to facilitate the growth of the cells.

A particularly preferred approach is to use perfusion of the cell culture medium. Perfusion refers to the introduction of medium into the bioreactor while removing medium from the bioreactor. It is particularly preferred that the new medium is introduced at the bottom of the bioreactor and the spent medium (e.g. comprising lactic acid) is removed from the top of the bioreactor, although the invention is not particularly limited in this regard. The new medium can be introduced by being pumped into the bioreactor. The spent medium may be removed by being pumped out of the bioreactor. The introduced medium may be fresh medium, in the sense it has not been previously introduced into the bioreactor and the removed medium can be spent medium, in the sense that it has been exposed to growing cells. However, the perfusion approach encompasses recirculating, at least part of, the medium through the bioreactor.

The bioreactor can regulate the temperature of the medium within which the cells are growing. This allows optimization of the cell growth.

The bioreactor may be any suitable size for culturing the cells. Accordingly, the bioreactor may have a capacity of at least 1 litre, or at least 2 litres, or at least 3 litres, or at least 5 litres, or at least 10 litres, or at least 20 litres, or at least 50 litres. The approach of the present invention is particularly useful for large scale processes. Accordingly, the bioreactor may be at least 100 litres, at least 250 litres, at least 500 litres, at least 1000 litres, or at least 2000 litres. A bioreactor system may be employed that contains a plurality of bioreactors, where each bioreactor is used to culture cells on its own substrate.

The particulate substrate may be of any suitable form that can be packed in an arrangement that operates as a packed bed. In other words, the substrate is in a particulate form that packs in a manner that maintains an open porosity allowing fluid flow through the packed bed within a bioreactor. The particulate substrate may be formed of a polymeric material, such as polyethylene terephthalate (PET), polystyrene, alginate, or agarose. The particulate substrate may be a hydrogel material. It may be particularly preferred that the particulate substrate is formed from an edible material, such as alginate.

Edible materials are those which can be safely eaten by humans and/or are considered safe for eating by humans, in line with the common understanding of edible products. Edible materials may comprise substantially, consist essentially of, or consist of materials designated Generally Recognized as Safe (GRAS) by the US Food and Drug Administration (FDA), in particular under sections 201(s) and 409 of the Federal Food, Drug, and Cosmetic Act (FDCA) and/or materials which comply with the Food Chemicals Codex (FCC). The edible materials may be digestible by humans. The edible materials may have nutritional value for humans.

Reference to a particulate substrate means that the substrate comprises a plurality of discrete particles that are not bound to each other so it is possible for the particles to move freely relative to each other.

The particulate substrates are preferably classed as macrocarriers. These particulate substrates typically pack in a manner that allows fluid to readily flow through the packed bed. In particular, the substrates may comprise, or consist of particles having a longest dimension of 1 mm or more, or 5 mm or more, or 10 mm or more. The substrate may comprise particles having a longest dimension of no less than 0.1 mm. The longest dimension refers to the longest dimension of the particle, for example, the longest dimension of a sphere is its diameter. Such macrocarriers are advantageous because they have a large surface area for cell growth and provide interstitial spaces between particles, allowing cell culture media to flow through a packed bed.

The particles may each be fibrous in nature. This increases the available area for cell growth and assists cell attachment.

Possible particulate substrates that can be used with the present invention include BioNOC™ II available from Esco VacciXcell.

The culturing of the cells occurs in the bioreactor with the flowing of cell culture medium through the packed bed such that the fixed bed conditions are maintained. In other words, the flow rate is such that it does not substantially disturb the arrangement of the particulate particles and the majority, i.e. greater than 50% of the particulate substrate by weight, remains in packed bed conditions, preferably greater than 75% of the particulate substrate by weight remains in packed bed conditions, even more preferably greater than 90% of the particulate substrate by weight remains in packed bed conditions, even more preferably greater than 95% of the particulate substrate by weight remains in packed bed conditions. Most preferably, substantially all, or all, of the particulate particles remain in packed bed conditions. This allows for some relative movement of the particles, but not to the extent that there is no longer the required fraction of particles in a packed bed. The use of macrocarriers particularly assists with the maintenance of the packed bed condition.

As used herein, a packed bed refers to a condition where each particle is in contact with all of the other particles, either directly or indirectly via other particles. The packed bed spans the full area of the bioreactor chamber within which it resides. In this way, the flowing fluid has to pass through the packed bed.

The maintenance of packed bed conditions reduces the forces (e.g. shear stress) experienced by the growing cells, thereby reducing the damage to and detachment of shear sensitive cells during the culturing process. Hence, the present invention is particularly suitable for use with shear sensitive cells, such as myoblasts.

The present invention also relates to a flow distributor. The flow distributor can be used with the method and the bioreactor described herein. The flow distributor can help distribute the cell culture medium uniformly within the bioreactor and thus help provide uniform growth conditions. This is particularly useful when operating under packed bed conditions.

The flow distributor can be positioned within the bioreactor such that the cell culture medium flows through the flow distributor before flowing through the packed bed.

This ensures that the flow through the packed bed is influenced by the flow distributor. The flow distributor may be the surface upon which the packed bed rests. In other words, it is the bottom of the chamber within which the particulate substrate is contained within the bioreactor. In this way the flow distributor has the greatest influence on the flow of the cell culture medium through the packed bed due to its proximity to the packed bed. The flow distributor may span the full area of the flow path within the bioreactor, accordingly, flowing fluid cannot by-pass the flow distributor and must past through it. This can be achieved by the flow distributor being shaped in a complementary manner to the internal shape of the bioreactor. For example, when the bioreactor has a cylindrical shape, the flow distributor may have a cylindrical shape such that it can be positioned within the bioreactor and span a complete circular cross-section of the bioreactor.

The flow distributor may be formed of any suitable material. For example, the flow distributor may be formed from a metallic material, such as stainless steel.

The flow distributor may be manufactured by any suitable technique. A particularly useful approach is additive manufacturing. This allows precise control over the form of the flow distributor.

The flow distributor may have a first surface and a second surface, wherein the first surface is on the opposite side of the flow distributor to the second surface. The first surface and second surface may be parallel surfaces, such as opposing ends of a cylinder. In particular, the first surface may be one circular end of a generally cylindrical flow distributor and the second surface may be the other circular end. The first surface and/or second surface may be generally planar. The first surface and/or second surface may comprise undulations. Such undulations may help influence how a packed bed rests on the surface.

The flow distributor is configured to allow fluid to flow through it. This can be achieved by the presence of a plurality of channels, each channel having an inlet and an outlet. It is intended for fluid to enter the channel at the inlet, travel through the channel and across the flow distributor and then exit at the outlet. The configuration of these channels can be used to impart desirable fluid flow characteristics on the exiting flow of fluid. Where the flow distributor has the first surface and the second surface described above, the channels may have their inlet on the first surface and their outlet on the second surface. The reference to inlets and outlets being on a surface refers to the presence of the respective opening within that surface.

The dimensions of the opening within the surface are preferably sufficiently small to prevent the passage of the particulate substrate into the opening. Accordingly, the particulate substrate comprises, or consists of, particles having a largest dimension that is greater than the largest dimension of the opening. This prevents loss of the particulate substrate from the relevant chamber in the bioreactor. It also means that a membrane filter is not required to confine the particulate substrate, since the flow distributor performs that function.

The flow distributor may have a central axis extending through it. This central axis is a reference point that can be used herein to define the orientations of the various channels. The central axis may extend through the centre of mass of the flow distributor.

It is preferred that the flow distributor has at least one axis of rotational symmetry. The axis of rotational symmetry with the highest order may be considered the central axis. Symmetry associated with the flow distributor contributes to its ability to provide a uniform distribution of fluid within the packed bed. In particular, when the flow distributor is cylindrical, the central axis is the longitudinal axis of the cylinder, i.e. perpendicular to the circular cross-section.

The plurality of channels within the flow distributor may be in any suitable arrangement to provide the desired fluid flow.

The plurality of channels may include channels that have outlets that are each arranged at a radial distance from the central axis, wherein these outlets are configured to direct the fluid at an angle towards the central axis relative to the tangent at the radial distance of that channel. In other words, the direction that the fluid is directed from the outlet has a component in a plane that is perpendicular to the central axis, and that component is at an angle towards the central axis relative to the tangent that is within that plane. This tangent is the tangent of a virtual circle that is centred on the central axis and has a radius to the outlet. Therefore, the tangent is the tangent of that circle at the point where the outlet is positioned on its circumference. Preferably, the fluid is directed at an angle greater than 5° towards the central axis relative to the tangent, more preferably greater than 10°. The fluid is directed at an angle of less than 80° relative to the tangent, and preferably less than 60°. By directing the exiting fluid towards the central axis it ensures that the exiting fluid flow continues through a large amount of the packed bed. This is in contrast to outlets that direct fluid along the tangent or at an angle away from the central axis, where the fluid will encounter a wall of the bioreactor sooner, thus reducing the influence of the flow distributor.

It is described herein how the outlets are configured to direct the fluid in particular directions. In order to achieve this, the length of the channels can be aligned along these directions. Accordingly, references to the directions that the fluid is directed in apply equally to the angle along which the channels run, i.e. the angle of the length of the channel relative to the reference. This angle can be constant along the full length of the channel.

It is stated herein that the outlets of the channels are configured to direct the exiting fluid in a certain manner. It should be understood that this refers to the configuration of the whole channel and not just the outlet. For example, the arrangement of the channel can ensure that the fluid that is exiting the outlet follows a certain path and is accordingly directed in a certain direction.

There may be channels that have a varying cross-sectional area along their length. The cross-sectional area is the area perpendicular to the length, while the length runs down the centre of mass of the channel (if the channel had uniform mass). A varying cross-sectional area can be used to vary properties of the exiting fluid.

There may be channels that have a first cross-sectional area at their inlet, a second cross-sectional area at their outlet and a third cross-sectional area at a point between their inlet and their outlet. The third cross-sectional area can be less than the first cross-sectional area and less than the second cross-sectional area. The first and second cross-sectional areas may be the same. Such an arrangement has been found to reduce the formation of stable bubbles by the presence of the increasing cross-sectional area. This is beneficial due to the damage that can occur to the shear sensitive cells when bubbles burst. The presence of the narrowing of the cross-sectional area also reduces the possibility of the particulate substrate falling through the flow distributor. Alternatively, the third cross-sectional area may be larger than the first cross-sectional area and larger than the second cross-sectional area. The cross-sectional areas at the inlet and at the outlet are the areas that are perpendicular to the channel length, while being fully contained within the channel, i.e., they are the last point along the length of the channel (towards the respective of the inlet or the outlet) where the cross-sectional area is fully defined by the channel walls.

A similar beneficial effect in relation to bubbles can also be achieved where there are channels that have a varying cross-sectional area that decreases from the outlet towards the inlet. In other words, the channel cross-sectional area tapers down from the outlet to the inlet.

A similar beneficial effect in relation to retaining substrate particles can also be achieved where there are channels that have a varying cross-sectional area that increases from the outlet towards the inlet. In other words, the channel cross-sectional area tapers down from the inlet to the outlet. This means the outlet is restricted in size resulting in a smaller opening into which substrate particles may fall.

In a further arrangement, the third cross-sectional area is greater than the first cross-sectional area and is greater than the second cross-sectional area. This can enable control of the size distribution of bubbles that are exiting the flow distributor.

The cross-sectional area throughout the channel may have a largest dimension of 10 mm or less, 5 mm or less, preferably 3 mm or less, even more preferably 2 mm or less. The cross-sectional area throughout the channel may be 1 mm or more. In particular, the first and second cross-sectional areas may have a largest dimension of 3 mm and the third cross-sectional area may have a largest dimension of 2 mm.

It is particularly preferred that the cross-sectional areas are circular, i.e. the channels are cylindrical.

There may be channels present that have an outlet that is configured to direct exiting fluid at an angle of between 10° and 80°, preferably between 20° and 60°, even more preferably between 30° and 45°, relative to a central axis direction of the flow distributor. Preferably, the central axis direction is parallel to the longitudinal axis of the bioreactor when the flow distributor is in place within the bioreactor. This means that the exiting fluid is not simply directed in a straight line through the flow distributor but it is given a component of flow that is angled away from the central axis direction. This provides for a more uniform distribution of fluid through the packed bed.

It should be noted that the plurality of channels may comprise channels having various combinations of properties described herein. For example, a set of channels may have channels with varying cross-sectional areas as well as being configured to direct the fluid flow in particular directions. Such combinations provide greater uniformity of the growth conditions. It is particularly preferred that there are channels present that each have an outlet that is configured to direct exiting fluid at an angle of between 10° and 50°, preferably between 30° and 45°, relative to a central axis direction of the flow distributor, as well as at an angle greater than 5° towards the central axis relative to the tangent and less than 80° relative to the tangent, and preferably less than 60°.

Further, the flow distributor may have channels that have different properties. For example, some of the channels may have a varying cross-sectional area, while others are configured to direct fluid in particular directions. This increases the flexibility associated with the flow distributor.

The plurality of channels may be arranged in any suitable manner within the flow distributor. In particular, the channels may be arranged such that the outlets are uniformly distributed across the surface of the flow distributor. However, alternative arrangements may be adopted.

The outlets of the plurality of channels may all be arranged to direct fluid in the same direction. This can include the same circumferential direction when the outlets are arranged in a circular arrangement around a central point, such as the central axis of the flow distributor. It has been found that a particularly good level of mixing is produced when the flow distributor has a plurality of channels where the outlets are configured to direct fluid in at least two different directions, for example, two different circumferential directions.

Overall, the use of a flow distributor as described herein helps to distribute the culture medium fluid in an effective manner despite the relatively low flow rates required to maintain the packed bed conditions. It is therefore a particularly useful approach for the delicate cells described herein.

Cultured cells may be removed from the particulate substrate. This may be done whilst the particulate substrate is still inside the bioreactor or after the particulate substrate has been removed from the bioreactor. The cultured cells may be removed using an enzymatic process, for example by trypsinization. The cultured cells may be removed using a chemical process, for example by treatment with Ca-and Mg-free PBS and EDTA. The cultured cells may be removed using a mechanical process, such as scraping. Where the particulate substrate comprises edible materials, it may not be necessary to disassociate the cells or all of the cells from the substrate. Nevertheless, it may be desirable to remove or reduce the amount of edible material in the final product, for example to improve texture. The use of alginate increases the ease of disassociating cells from the substrate after they have been cultured on the substrate. In particular, disassociation of cells from alginate can be achieved by using alginate-lyase to break down the alginate. Any appropriate digestion enzyme can be utilised depending on the material used to form the (edible) materials.

In the invention, the cultured cells typically include muscle cells, e.g. myocytes and/or myotubes. Once removed from the substrate, these cells may be used directly as a meat product for consumption. Alternatively, the cells may be biomass which is further processed into a final meat product for consumption. Thus, the methods of the invention may further comprise a step of processing the cells into a meat product for consumption, e.g. for human consumption.

The cultured cells may be used as a replacement for livestock-derived meat in a meat product for consumption using suitable food production techniques as are well known to the skilled person. For example, the cultured cells may be simply added into a mixture intended to form meatballs or sausages as a replacement or addition to livestock-derived meat, and the mixture then processed into the meat product using conventional techniques.

As used herein, the term “polymer” refers to oligomers and both homopolymers and copolymers, and the prefix “poly” refers to two or more.

Singular encompasses plural and vice versa. For example, although reference is made herein to “a” particulate substrate, “a” flow distributor, and the like, one or more of each of these and any other components can be used.

The terms “comprising” and “comprises” as used herein are synonymous with “including”, “includes” or “containing”, “contains”, and are inclusive or open-ended and do not exclude additional, non-recited members, elements or method steps.

Additionally, although the present invention has been described in terms of

“comprising”, the invention as detailed herein may also be described as “consisting essentially of” or “consisting of”.

Although the present invention has been described in terms of “obtainable by”, the associated features of the present invention detailed herein may also be independently described as “obtained by”.

As used herein, the term “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

Where ranges are provided in relation to a genus, each range may also apply additionally and independently to any one or more of the listed species of that genus.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

For a better understanding of the invention, and to show how the invention may be carried into effect, reference will now be made, by way of example, to the following figures and experimental data.

### FIGURES

FIG. 1 depicts a schematic arrangement for putting the present invention into effect;

FIG. 2a depicts a plan view of a flow distributor of the present invention, the depiction is a wire frame depiction so the channels within the flow distributor can be seen;

FIG. 2b depicts a perspective view of the flow distributor of FIG. 2a;

FIG. 2c depicts a side view of the flow distributor of FIG. 2a;

FIG. 3a depicts a plan view of a further flow distributor, the depiction is a wire frame depiction so the channels within the flow distributor can be seen;

FIG. 3b depicts a perspective view of the flow distributor of FIG. 3a;

FIG. 3c depicts a side view of the flow distributor of FIG. 3a;

FIG. 3d depicts a detail view of channels within the flow distributor of FIG. 3a;

FIG. 4a depicts a plan view of another flow distributor, the depiction is a wire frame depiction so the channels within the flow distributor can be seen;

FIG. 4b depicts a perspective view of the flow distributor of FIG. 4a;

FIG. 4c depicts a side view of the flow distributor of FIG. 4a;

FIG. 4d depicts a detail view of channels within the flow distributor of FIG. 4a.

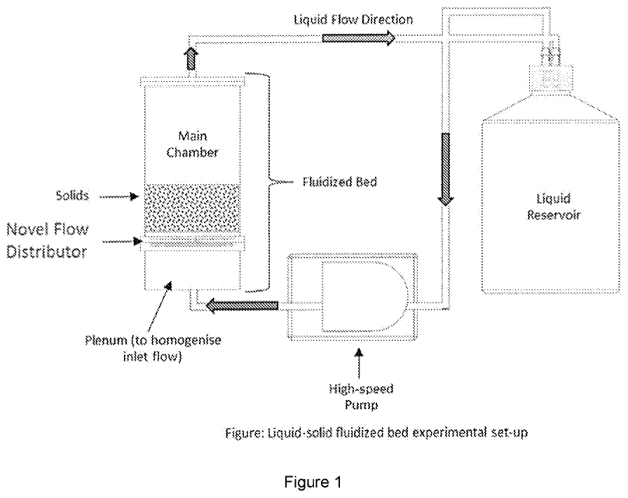

FIG. 1 depicts a schematic arrangement demonstrating the presence of a flow distributor (denoted ‘Novel Flow Distributor’) in a bioreactor system. The particulate substrate (denoted ‘Solids’) rests on the flow distributor within the bioreactor (denoted ‘Fluidized Bed’). The flow of cell culture medium into the bioreactor is achieved by a pump (denoted ‘High-speed Pump’). This flows through the flow distributor first and then through the particulate solids. The flow rate is controlled so that the particulate solids remain as a packed bed during the culturing process.

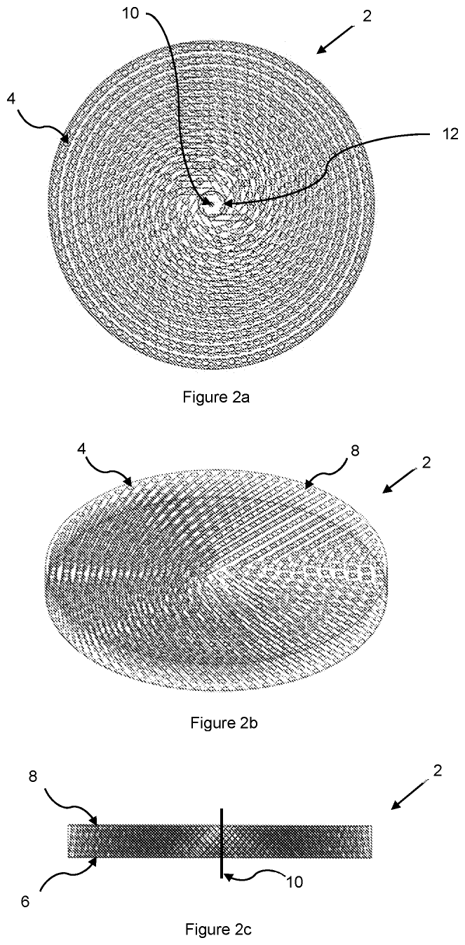

FIGS. 2a-c depict a flow distributor 2 that has a plurality of channels 4. The channels 4 each have an inlet on the first surface 6 and an outlet on the second surface 8. The flow distributor 2 is cylindrical in shape where the first surface 6 and second surface 8 are circular end faces of the cylinder. The flow distributor 2 has a central axis 10 that is the longitudinal axis of the cylindrical shape of the flow distributor 2. This central axis is not actually a physical part of the flow distributor it is merely superimposed on the image due to its use as a reference axis. This central axis can be used as the reference point for describing the configuration of the channels 4. As can be seen from FIG. 2a, the outlets of the channels 4 are arranged on the second surface as a plurality of concentric circles. The innermost circle of these concentric circles is highlighted on FIG. 2a and denoted 12. This circle 12 is not actually present on the flow distributor but it has been superimposed on FIG. 2a to aid the explanation of the arrangement. It can be seen that the outlets are arranged on this first circle. Therefore, each of these outlets is at a radial distance from the central axis 10. This radial distance is the radius of the virtual circle 12. The path of each of the channels to these outlets is also visible. As can be seen from the plan view of FIG. 2a, the channels extend along the direction of the tangent of the circle 12 at the point of the outlet. Accordingly, these channels are configured to direct fluid along the tangent at the radial distance of the outlet of that channel. As noted herein, it is preferable that the channels are actually configured to direct fluid at an angle towards the central axis 10 relative to this tangent in order to increase the distance the projected fluid travels before encountering a wall of the bioreactor.

As can be appreciated from FIGS. 2b and 2c, the channels are configured to direct exiting fluid at an angle relative to the central axis 10. In this case the channels are angled at 45° to the central axis direction, i.e. the direction along which the central axis runs, this provides an exiting fluid that is directed at an angle of 45° relative to the central axis 10 of the flow distributor 2. The outlets are all arranged to direct fluid in the same clockwise circumferential direction.

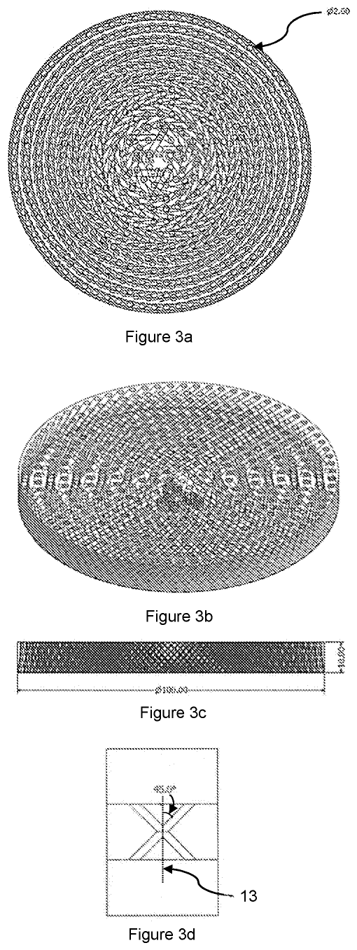

A similar arrangement is depicted in FIGS. 3a-d, apart from this time alternative concentric circles of outlets are configured to direct fluid in alternative circumferential directions, the outermost directs the fluid clockwise, while the next outermost directs the fluid anti-clockwise, which alternates with each concentric circle. Such an arrangement can increase the amount of mixing of the exiting fluid.

FIG. 3d depicts two of the channels in adjacent rings with lengths running so as to direct fluid in circumferentially opposite directions. It is also depicted how these channels are arranged at 45° to the central axis direction 13.

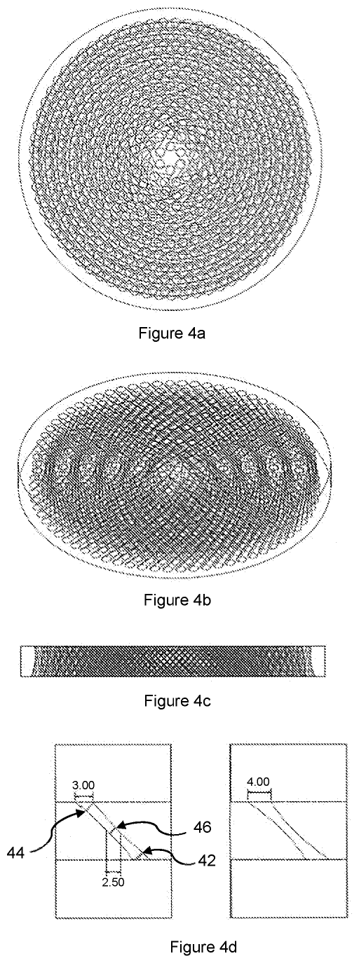

A further arrangement is depicted in FIGS. 4a-d. This arrangement is similar to the arrangement depicted in FIGS. 3a-d, apart from this time having channels with varying cross-sectional areas. This can be seen in more detail in FIG. 4d. Here a cross-section through a channel is depicted that shows a channel having a first cross-sectional area at the inlet 42, a second cross-sectional area at the outlet 44 and a third cross-sectional area at a point 46 between the inlet 42 and the outlet 44. This third cross-sectional area is less than the first and second cross-sectional areas. As can be seen, the first cross-sectional area is the last point along the length of the channel towards the inlet where the cross-sectional area is fully contained by the channel. Further, the second cross-sectional area is the last point along the channel towards the outlet where the cross-sectional area is fully contained by the channel.

### EXAMPLES

The ability of the method and apparatus of the present invention to culture cells was tested using apparatus according to FIG. 1.

### Example 1

A main chamber containing a PET macroporous particulate substrate packed bed was fluidly connected downstream from a flow distributor according to FIGS. 2a-c. The surface area of the particulate substrate in the main chamber was 8,000 cm² and the working volume of the main chamber was 150 ml.

During the test period a DMEM/F-12 (Dulbecco's Modified Eagle Medium/Nutrient Mixture F-12) culture media was pumped into the main chamber through the flow distributor. The flow rate was controlled to induce a superficial liquid velocity of around 1.7 cm/min so that substantially all of the particulate substrate remained as a packed bed during the culturing process.

19 million primary porcine myoblasts were seeded on day 0. The day 1 seeding efficiency was 97%. The day 8 cell count was 0.65 billion cells with a cell surface density of 81,250 cell/cm².

A 34-fold expansion and 4.3×10⁶ cells/ml process intensification was achieved, with a doubling time of 38 hours.

### Example 2

A main chamber containing a PET macroporous particulate substrate packed bed was fluidly connected downstream from a flow distributor according to FIGS. 2a-c. The surface area of the particulate substrate in the main chamber was 32,000 cm² and the working volume of the main chamber was 900 ml.

During the test period a DMEM/F12 culture media was pumped into the main chamber through the flow distributor. The flow rate was controlled to induce a superficial liquid velocity of around 1.7 cm/min so that substantially all of the particulate substrate remained as a packed bed during the culturing process.

76.8 million primary porcine myoblasts were seeded on day 0. The day 1 seeding efficiency was 92%. The day 8 cell count was 1.4 billion cells with a cell surface density of 43,750 cell/cm².

An 18-fold expansion and 1.6×10⁶ cells/ml process intensification was achieved, with a doubling time of 46 hours.

The results achieved by Examples 1 and 2 were compared to comparative methods and apparatus as follows.

### Comparative Example 1

A stirred tank bioreactor main chamber containing a polystyrene microcarrier particulate substrate and 100 ml of a DMEM/F12 culture media was used.

1 million primary porcine myoblasts were seeded on day 0. The day 10 cell count was 40 million cells with a cell surface density of 40,000 cells/cm².

A 40-fold expansion and 0.4×10⁶ cells/ml process intensification was obtained, with a doubling time of 45 hours.

### Comparative Example 2

A stacked culture chamber with tissue culture (TC)-treated surfaces was used with a DMEM/F12 culture media.

50 million primary porcine myoblasts were seeded on day 0. The day 4 cell count was 0.87 billion cells with a cell surface density of 137,000 cells/cm².

A 20-fold expansion and 0.73×10⁶ cells/ml process intensification was obtained, with a doubling time of 22 hours.

1. A method of culturing cells comprising
providing a bioreactor, wherein the bioreactor contains a particulate substrate arranged as a fixed bed;
seeding the particulate substrate with myoblasts, myocytes, and/or myotubes;
culturing the cells by flowing cell culture medium through the fixed bed such that the fixed bed conditions are maintained.

2. The method of claim 1, further comprising a flow distributor within the bioreactor, wherein the flow distributor comprises a plurality of channels, wherein each of the plurality of channels has an inlet and an outlet, wherein the cell culture medium flows through the flow distributor, from the channel inlets to the channel outlets, before the flowing of the cell culture medium through the fixed bed.

3. A flow distributor for a bioreactor comprising
a first surface and a second surface, wherein the first surface is on the opposite side of the flow distributor to the second surface, and wherein the flow distributor has a central axis extending through the flow distributor; and
a plurality of channels within the flow distributor, wherein the plurality of channels each have an inlet and an outlet, wherein the inlet is on the first surface and the outlet is on the second surface;
wherein the plurality of channels comprises channels that have outlets that are each arranged at a radial distance from the central axis, wherein these outlets are configured to direct the fluid at an angle towards the central axis relative to the tangent at the radial distance of the outlet of that channel.

4. The method of claim 2, wherein the plurality of channels comprises channels having a varying cross-sectional area along their length.

5. The method of claim 4, wherein the channels having a varying cross-sectional area along their length comprise channels having a first cross-sectional area at their inlet, and a second cross-sectional area at their outlet and a third cross-sectional area at a point between their inlet and their outlet, wherein the third cross-sectional area is less than the first cross-sectional area and less than the second cross-sectional area.

6. The method of claim 4, wherein the channels having a varying cross-sectional area have a cross-sectional area that decreases from the outlet towards the inlet.

7. The method of claim 2, wherein the plurality of channels comprises channels with an outlet configured to direct exiting fluid at an angle of between 10° and 80° relative to a central axis direction of the flow distributor.

8. The method of claim 2, wherein the plurality of channels comprises channels that have outlets that are each arranged at a radial distance from a central axis extending through the flow distributor, wherein these outlets are configured to direct the fluid at an angle towards the central axis relative to the tangent at the radial distance of the outlet of that channel.

9. The method of claim 1, further comprising a step of processing the cells into a meat product for consumption.

10. A bioreactor system for culturing cells comprising
a bioreactor; and
a flow distributor according to claim 3 within the bioreactor.

11. The bioreactor system of claim 10, further comprising a particulate substrate within the bioreactor.

12. The bioreactor of claim 10, wherein the cells are myoblasts, myocytes, and/or myotubes.

13. (canceled)
